(19) United States
(12) Patent Application Publication
(10) Pub. No.: US 2018/0228648 A1
(43) Pub. Date: Aug. 16, 2018

(54) METHOD AND OPHTHALMIC DEVICE WITH ACTIVE AGENT RELEASE SYSTEM

(71) Applicant: Johnson & Johnson Vision Care, Inc., Jacksonville, FL (US)

(72) Inventors: Randall B Pugh, Jacksonville, FL (US); Anthony W Martin, Jacksonville, FL (US); Sharika Snook, Jacksonville, FL (US)

(21) Appl. No.: 15/951,144

(22) Filed: Apr. 11, 2018

Related U.S. Application Data
(62) Division of application No. 14/696,126, filed on Apr. 24, 2015.
(60) Provisional application No. 61/984,590, filed on Apr. 25, 2014.

(51) Int. Cl. A61F 9/00; A61M 31/00

(52) U.S. Cl. CPC A61F 9/0017; A61M 2205/3584; A61F 2250/0002; A61M 2205/50; A61F 2250/0068; A61M 2205/3538; A61M 31/002; A61M 2205/3507

(57) ABSTRACT

The present invention provides an energized ophthalmic device with an active agent release system and an associated method. The active agent release system can be suitable to dispense an active agent including, for example, a vitamin, lubricant, a saline, a solvent, and/or medicament, at one or more predetermined times, through the use of an energization element contained in the ophthalmic device. The energization element may be a battery and/or an energy receptor antenna. The release of the active agent can be according to a signal received wirelessly, a predetermined time, and/or a sensed condition, which can cause an activation element to conduct a current to at least a portion of a metal cap under stress causing it to fold and thereby expose the active agent to a surrounding environment.

### CLAIM OF PRIORITY

The present application is a divisional of U.S. patent application Ser. No. 14/696,126 filed Apr. 24, 2015, which claims priority to provisional U.S. Patent Application No. 61/984,590, filed Apr. 25, 2014, which is incorporated by reference in its entirety.

### FIELD OF THE INVENTION

The present invention provides an energized ophthalmic device including an array of containment cells, wherein each containment cell includes a cap that can be actuated electrically by a cell activation element to release an active agent contained within each of the containment cells.

### BACKGROUND OF THE INVENTION

Active agents are frequently administered to the eye for the treatment of ocular diseases and disorders. Conventional means for delivering active agents to the eye involve topical application to the surface of the eye. The eye is uniquely suited to topical administration because, when properly constituted, topically applied active agents can provide lubrication and/or penetrate through the cornea and rise to therapeutic concentration levels inside the eye. Active agents for ocular diseases and disorders may be administered orally or by injection, but such administration routes can be disadvantageous in that, in oral administration, the active agent may reach the eye in too low a concentration to have the desired pharmacological effect, and their use can be complicated by significant, systemic side effects and injections pose the risk of infection.

The majority of ocular active agents and/or lubricants are currently delivered topically using eye drops which, though effective for some applications, can be inefficient. When a drop of liquid is added to the eye, it overfills the conjunctival sac, the pocket between the eye and the lids, causing a substantial portion of the drop to be lost due to overflow of the lid margin onto the cheek. In addition, a substantial portion of the drop that remains on the ocular surface is drained into the lacrimal puncta, diluting the concentration of the drug.

To compound the problems described above, patients often do not use their eye drops as prescribed. Often, this poor compliance is due to an initial stinging or burning sensation caused by the eye drop. Certainly, instilling eye drops in one's own eye can be difficult, in part because of the normal reflex to protect the eye. Therefore, sometimes one or more drops miss the eye. Older patients may have additional problems instilling drops due to arthritis, unsteadiness, and decreased vision. Pediatric and psychiatric patient populations pose difficulties as well.

Prior topical sustained release systems include gradual release formulations, either in solution or ointment form, which are applied to the eye in the same manner as eye drops but less frequently. Such formulations are disclosed, for example, in U.S. Pat. No. 3,826,258 issued to Abraham and U.S. Pat. No. 4,923,699 issued to Kaufman. Due to their method of application, however, these formulations result in many of the same problems detailed above for conventional eye drops. In the case of ointment preparations, additional problems are encountered such as a blurring effect on vision and the discomfort of the sticky sensation caused by the thick ointment base.

Alternately sustained release systems have been configured to be placed into the conjunctival cul-de-sac, between the lower lid and the eye. Such units typically contain core drug-containing containment cells surrounded by a hydrophobic copolymer membrane which controls the diffusion of the drug. Examples of such devices are disclosed in U.S. Pat. No. 3,618,604 issued to Ness, U.S. Pat. No. 3,626,940 issued to Zaffaroni, U.S. Pat. No. 3,845,770 issued to Theeuwes et al., U.S. Pat. No. 3,962,414 issued to Michaels, U.S. Pat. No. 3,993,071 issued to Higuchi et al., and U.S. Pat. No. 4,014,335 issued to Arnold. However, due to their positioning, the units may be uncomfortable and poor patient acceptance is again encountered. Moreover, leakage of the active agent should be prevented when some active agents are used. Specifically, when administering active agents, the effectiveness of the active agent may be compromised when the active agent receptors are exposed to them continuously.

Other methods similarly allow for the eluting of an active agent, e.g., medicament and/or a lubricant, over a period of time. Again, some active agents however can be most efficacious when periodically delivered in a predetermined dosed amount or at a time of need. In one approach seeking to provide delivery of an active agent at pre-determined times, a containment device with multi-layer reservoir cap structure has been described in U.S. Pat. No. 8,211,092, issued to Uhland et al. This system however uses an electrical current to rupture, i.e., melt or vaporize, a reservoir's cap using the heat generated by the electrical current. Although the described delivery system may be suitable for the delivery of an active agent in some environments, this system would generally not be suitable for use in sensitive organs or environments, including, for example, an ophthalmic environment, due to the flash and heat generated during rupture of the cap which can damage surrounding cells. Further, the described system may also not be suitable in a sensitive organ or environment as the rupture will produce debris that can damage or bother the surrounding organ or environment. In an ophthalmic environment, for example, the debris may detrimentally affect the vision of a user.

Accordingly, alternative methods, systems, and devices for delivering medicaments to an ophthalmic area may be beneficial especially if discrete dosage amounts may be delivered over significant periods of time in a way that is innocuous to the user.

### SUMMARY OF THE INVENTION

In one aspect, the present invention provides an energized ophthalmic device incorporating an active agent release system. According to some aspects, the active agent release system can be suitable to dispense an active agent through the use of an energizing element capable of energizing an activation element that is configured to energize and cause a metal cap assembled under stress to fold upon energization.

According to some aspects of the present invention, an active agent release system can include a substrate having one or more containment cells. At least one of the one or more containment cells can contain an active agent enclosed by a metal cap bonded under stress to a surface of the substrate. An energy source can be in electrical connection to an activation element configured to conduct an electrical current to at least a portion of the metal cap upon receipt of an activation signal. The electrical current and the stress cause the metal cap to fold and expose the active agent to a surrounding environment.

According to additional aspects of the invention, an active agent dispensing digital microarray device comprising is provided. The digital microarray can be used, for example, in an ophthalmic device and can include a semiconductor material substrate including one or more containment cells, at least one of the one or more containment cells containing an active agent enclosed by a biocompatible metal cap bonded to a surface of the semiconductor material substrate under stress; and a micro-processor energized by an energy source and in connection with an activation element. The activation element can be configured to conduct electricity to at least a portion of the biocompatible metal cap upon the receipt of an activation signal from the micro-processor. The stress of the biocompatible metal cap and the electricity conducted are sufficient to cause the biocompatible metal cap to fold and expose the active agent contained in at least one of the one or more containment cells.

In yet additional aspects, an associated method of using the active agent release system is provided. The method includes forming a substrate having one or more containment cells; depositing one or more active agents into at least one of the one or more containment cells; forming a hermetic seal over an opening of at least one of the one or more containment cells by bonding a biocompatible metal cap under stress to a surface of the substrate; and providing an activation element configured to conduct electricity from an energy source to at least a portion of the biocompatible metal cap causing the biocompatible metal cap to fold and expose the active agent to a surrounding environment.

The active agent release system can be controlled by a micro-processor in the ophthalmic device and be in electrical connection with the energy source and an antenna. The micro-processor can be configured to perform a variety of functions related to the control and activation of the active agent release system. For example, the micro-processor can be configured to wirelessly receive, using the antenna, one or more activation signals from a device. The device can include, for example, a smart phone, a tablet, a personal computer, a remote transmitter, and a medical drug delivery controller device, and may communicate with the micro-processor of the ophthalmic device via one or more suitable LAN and/or WAN type radio or electromagnetic technology, preferably low power technologies.

Further, in some embodiments, the system micro-processor can be in electrical connection with one or more sensor(s) of said digital microarray and configured to be energized by the energy source. The one or more sensor(s) can include, for example, a biosensor configured to measure the concentration of one or more biomarkers contained in ocular fluid and to send an activation signal when the measured concentration falls outside a predetermined threshold. For example, at least one of the one or more sensors can be configured to determine when the ocular surface is above a comfortable dryness level and dispense a lubricant to the ophthalmic environment when it is needed. In addition or alternatively, in some embodiments, a timing element can be configured to provide the activation signal for a current to cause at least one of the one or more metal caps to deform and allow dispensing of the active agent at one or more pre-determined time(s).

The metal cap can form a hermetic seal over an opening of the one or more containment cells. In preferred embodiments that can deliver multiple doses, multiple metal caps may be arranged so that each metal cap covers an opening of a containment cell. Each metal cap can be under stress by the bonding nature during production or through the use of a binary shape memory alloy. For example, the metal cap can include one or more biocompatible metals including: gold, titanium, nickel, stainless steel, cobalt-chromuim, and nitinol. The active agent can include one or more of: a lubricant, a saline, a solvent, a pharmaceutical (e.g., a medicament), and a nutraceutical (e.g, a vitamin).

There has thus been outlined, rather broadly, certain aspects of the invention in order that the detailed description provided herein may be better understood, and in order that the present contributions to the art may be better appreciated. There are, of course, additional aspects of the invention that will be described below and which will illustrate the subject matter of the claims appended hereto.

In this respect, it is to be understood that the invention is not limited in its application to the details of construction and to the arrangements of the components set forth in the following description or illustrated in the drawings. The invention is capable of aspects in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the invention. It is important, therefore, that the claims be regarded as including such equivalent constructions insofar as they do not depart from the spirit and scope of the invention.

### BRIEF DESCRIPTION OF THE DRAWINGS

The foregoing and other features and advantages of the invention will be apparent from the following, more particular description of preferred embodiments of the invention, as illustrated in the accompanying drawings.

FIG. 1A is a diagrammatic representation of the top view of a media insert that may be included as part of an ophthalmic device including both optics and the active agent release system in accordance with aspects of the present invention.

FIG. 1B is a diagrammatic representation of an isometric view of an ophthalmic device including the media insert depicted in FIG. 1A including both optics and the active agent release system in accordance with aspects of the present invention.

FIG. 2 is a close up representation of active agent release features in an energized containment array that may be incorporated in an ophthalmic device in accordance with aspects of the present invention.

FIG. 3 is a schematic diagram of an exemplary cross section of stacked die integrated components implementing the active agent release system in accordance with aspects of the present invention.

FIG. 4 illustrates an exemplary assembly flow for assembling an energization source with electronics and a containment array into the ophthalmic device.

FIG. 5 is a schematic diagram of an exemplary micro-processor that may be used to implement some aspects of the present invention.

FIG. 6 illustrates an exemplary design for interconnections to individual active agent containers in a containment array.

FIG. 7 illustrates a block diagram of an ophthalmic device with an energized containment array.

FIG. 8 is a flow chart with exemplary steps that may be used to carry out aspects of the present invention.

### DETAILED DESCRIPTION OF THE INVENTION

The disclosure will now be described with reference to the drawing figures, in which like reference numerals can refer to like parts throughout.

Various aspects of the ophthalmic device and method disclosed may be illustrated by describing components that are coupled, bonded, sealed, attached, and/or joined together. As used herein, the terms “coupled”, “bonded”, “sealed”, “attached”, and/or “joined” are used to indicate either a direct connection between two components or, where appropriate, an indirect connection to one another through intervening or intermediate components. In contrast, when a component is referred to as being “directly coupled”, “directly bonded”, “directly sealed”, “directly attached”, and/or “directly joined” to another component, there are no intervening elements present.

Relative terms such as “lower” or “bottom” and “upper” or “top” may be used herein to describe one element's relationship to another element illustrated in the drawings. It will be understood that relative terms are intended to encompass different orientations in addition to the orientation depicted in the drawings. By way of example, if aspects of an exemplary ophthalmic device shown in the drawings are turned over, elements described as being on the “bottom” side of the other elements would then be oriented on the “top” side of the other elements. The term “bottom” can therefore encompass both an orientation of “bottom” and “top” depending on the particular orientation of the apparatus.

Various aspects of an ophthalmic device with an active agent release system may be illustrated with reference to one or more exemplary embodiments. As used herein, the term “exemplary” means “serving as an example, instance, or illustration,” and should not necessarily be construed as preferred or advantageous over other embodiments disclosed herein.

### Glossary

In this description and claims directed to the disclosed invention, various terms may be used for which the following definitions will apply:

Active agent: as used herein refers to an agent capable of treating, inhibiting, or preventing a disorder or a disease, and/or enhancing the physiological performance of cells or tissues. Exemplary active agents include, without limitation, a lubricant, a saline, a solvent, a pharmaceutical (e.g., a medicament), and a nutraceutical (e.g., a vitamin). In some embodiments, preferred active agents can be capable of lubricating and/or treating, inhibiting, or preventing a disorder or a disease of one or more of the eye, nose, and throat. The lubricants, for example, may be used to facilitate or inhibit cell wall permeability.

Energize(d): as used herein refers to the state of: being able to supply electrical current, having electrical energy applied to, or having electrical energy stored within.

Energy: as used herein refers to the capacity of a physical system to do work. Many uses within this disclosure may relate to the said capacity being able to perform electrical actions in doing work.

Energy source: as used herein refers to a device or layer that is capable of supplying energy or placing a logical or electrical device in an energized state.

Energy harvester: as used herein refers to a device capable of extracting energy from the environment and converting it to electrical energy.

Functionalized: as used herein refers to making a layer or device able to perform a function including for example, energization, activation, or control. In some embodiments, the function of the layer and/or the device may be used to provide various tasks including, for example, a chemical reaction, a change of surface properties, or to provide an ionic charge.

Ophthalmic device: as used herein refers to any device that resides in or on the eye. These devices may provide optical correction, vision enhancement, may be cosmetic, and/or may provide functionality unrelated to the eye. For example, the term “lens” may refer to a contact lens, overlay lens, ocular insert, optical insert, or other similar device through which vision is corrected or modified, or through which eye physiology is cosmetically enhanced (e.g. iris color). Alternatively, the lens may provide non-optic functions such as the functions described including, for example, monitoring biomarkers, delivering signals and/or administering active agents.

Lithium ion cell: as used herein refers to an electrochemical cell where lithium ions move through the cell to generate electrical energy. This electrochemical cell, typically called a battery, may be reenergized or recharged in its typical forms.

Media insert: as used herein refers to an encapsulated insert that will be included in an energized ophthalmic device. The energization elements and circuitry may be incorporated in the media insert. The media insert can define the primary purpose of the energized ophthalmic device. For example, in embodiments where the energized ophthalmic device allows the user to adjust the optic power, the media insert may include energization elements that control a liquid meniscus portion, or a liquid crystal portion, in the optical zone. Alternatively, a media insert may be annular so that the optical zone is void of material. In such embodiments, the energized function of the lens may not be optic quality but may be, for example, light polarization, photochromic functionality, color change, monitoring glucose, sound delivery, and/or administering medicine.

Operating mode: as used herein refers to a current draw state where the current over a circuit allows the device to perform its primary energized function.

Optical power: as used herein refers to the optical properties of an optical element including, for example, an ophthalmic lens.

Optical zone: as used herein refers to an area of an ophthalmic lens through which a wearer of the ophthalmic lens sees.

Power: as used herein refers to work done or energy transferred per unit of time.

Rechargeable or re-energizable: as used herein refers to a capability of being restored to a state with higher capacity to do work. Many uses may relate to the capability of being restored with the ability to flow electrical current at a certain rate and for a certain, reestablished period.

Reenergize or recharge: as used herein refers to restoring to a state with higher capacity to do work. Many uses may relate to restoring a device to the capability to flow electrical current at a certain rate and for a certain, reestablished period.

Reset function: as used herein refers to a self-triggering algorithmic mechanism to set a circuit to a specific predetermined state, including, for example, logic state or an energization state. A reset function may include, for example, a power-on reset circuit, which may work in conjunction with the switching mechanism to ensure proper bring-up of the chip, both on initial connection to the power source and on wakeup from storage mode.

Sleep mode or standby mode: as used herein refers to a low current draw state of an energized device after the switching mechanism has been closed that allows for energy conservation when operating mode is not required.

Stacked: as used herein means to place at least two component layers in proximity to each other such that at least a portion of one surface of one of the layers contacts a first surface of a second layer. In some embodiments, a film, whether for adhesion or other functions may reside between the two layers that are in contact with each other through said film.

Stacked integrated component devices or SIC devices: as used herein refers to the products of packaging technologies that assemble thin layers of substrates that may contain electrical and electromechanical devices into operative-integrated devices by means of stacking at least a portion of each layer upon each other. The layers may include component devices of various types, materials, shapes, and sizes. Furthermore, the layers may be made of various device production technologies to fit and assume various contours.

Storage mode: as used herein refers to a state of a system including electronic components where a power source is supplying or is required to supply a minimal designed load current. This term is not interchangeable with standby mode.

Substrate insert: as used herein refers to a formable or rigid substrate capable of supporting an energy source and/or a series of containment cells within an ophthalmic lens. In some embodiments, the substrate insert also supports one or more components.

Switching mechanism: as used herein refers to a component integrated with the circuit providing various levels of resistance that may be responsive to an outside stimulus, which is independent of the ophthalmic device.

In the past few decades, ophthalmic lenses have been improved to help treat conditions of dry eye, among others. More recently they have gained attention for use as drug delivery systems for the treatment of ocular diseases and conditions. However, as previously mentioned, several challenges exist with formulating a drug to release at the desired daily rate and/or dose that will give efficacy while limiting adverse events. According to some aspects of the present invention, an alternative or supplementary release strategy can involve the use of energized micro-electronics to control and enact the innocuous delivery of individual dose amounts at pre-determined times, upon demand and/or upon a sensed condition.

Unlike diffusion based delivery systems, which are characterized by a release rate which is dependent on the active agent diffusing through an inert water insoluble membrane barrier, the present invention can allow for delivery of an active agent upon demand, addressing shortcomings of diffusion based drug delivery and leaking. For example, there are two basic diffusion designs: reservoir devices and matrix devices. Reservoir devices are those in which a core of drug is surrounded by a polymeric membrane. The nature of the membrane determines the rate of release of drug from the system and there is often leakage throughout. The process of diffusion is generally described by a series of equations governed by Fick's first law of diffusion. A matrix device typically consists of a drug dispersed homogenously throughout a polymer. Both of these provide constant exposure by a tissue surface which may include the receptors to the active agent, e.g., a drug. By exposing tissue constantly to the active agent, the efficacy of the active agent can decrease over time, and in some events, prevent the active agent from having the intended effect completely.

Accordingly, reservoir and matrix drug delivery systems are considered diffusion based sustained release systems and constitute any dosage form that provides continuous medication over a period of time, often an extended period of time. The intended goal of a sustained release system is to maintain therapeutic levels of a drug for an extended period and this is usually accomplished by attempting to obtain zero-order release from the sustained release system. Sustained release systems generally do not attain this type of release profile but try to approximate it by releasing in a slow first-order manner. Over time, however, the drug release rate from reservoir and matrix sustained release systems will decay and become non therapeutic.

Recent developments in ophthalmic devices including, for example, contact lenses, have occurred enabling functionalized ophthalmic devices that can be energized. The energized ophthalmic device can include the necessary elements to correct and/or enhance the vision of users using embedded micro-electronics. Additional functionality using micro-electronics can include, for example, variable vision correction, tear fluid analysis, audio, and/or visual feedback to the user. According to some aspects of the present invention, an ophthalmic device that can include an active agent release system that can be capable of releasing an active agent to the ophthalmic environment of a user, upon demand, at a pre-determined time, and/or upon a sensed condition, is provided. The release can be generally innocuous to the user or in some embodiments allow for simple participation by the user. For example, one or more active agent(s) may be contained in one or more containment cells, each preferably enclosed by a metal cap that is bonded, under stress, to seal each one of the containment cells until an activation element is engaged. In some embodiments, a processor forming part of the active agent release system can be in wireless communication with one or more device(s) and receive signal data that can be used for the release of the active agent. The device(s) can include, for example, a smart phone, a tablet, a personal computer, a remote transmitter (e.g., a fob, MP3 player, or PDA), and a medical drug delivery device (e.g., a drug pump), and the like.

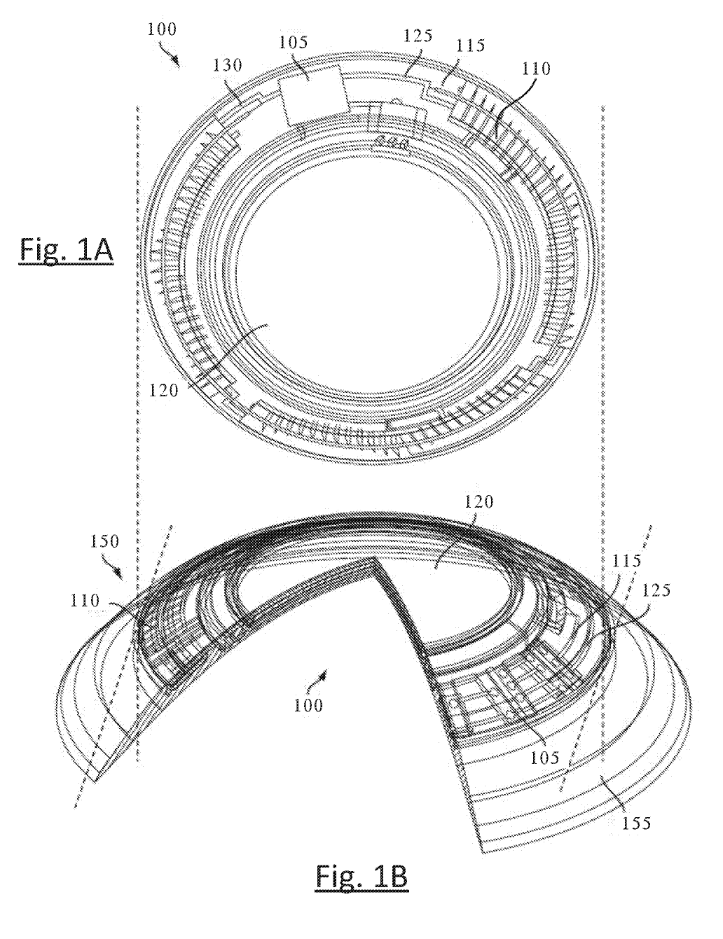

Referring now to FIG. 1A, a diagrammatic representation of the top view of a media insert that may be included as part of an exemplary ophthalmic device including both optics and an active agent release system is depicted. In particular, FIG. 1A shows a top view of an exemplary media insert 100 for an energized ophthalmic device 150 (shown in FIG. 1B) that includes the active agent release system 105. In some embodiments, the media insert 100 includes an optical zone 120 that may or may not be functional to provide vision correction. In embodiments where the energized function of the ophthalmic device is unrelated to vision, the optic zone 120 of the media insert 100 may be void of material. The media insert 100 can include a portion outside of the optical zone 120 including a substrate 115 incorporated with energization elements 110 connected to electronic components, including the active agent release system 105, by a series of interconnects, e.g., 125 and 130. In alternative embodiments, some electronic components may be included in the optical zone without detrimentally affecting the overall intended optical properties of the ophthalmic device. In such embodiments, for example, the electronic components may have translucent properties, be located in the center, or be small enough to not impact the overall intended optical effect.

Referring now to FIG. 1B, a diagrammatic cross section representation of an energized ophthalmic device 150 with the media insert 100 including both optics and the active agent release system 105 of FIG. 1A is depicted. According to some aspects of the present invention, the ophthalmic device 150 may be a contact lens designed to rest on the anterior surface of a patient's eye. For example, ophthalmic lens 100 may include a soft hydrogel skirt 155 which can include a silicone-containing component. A “silicone-containing component” is one that contains at least one [—Si—O—] unit in a monomer, macromer or prepolymer. Preferably, the total Si and attached O are present in the silicone-containing component in an amount greater than about 20 weight percent, and more preferably greater than 30 weight percent of the total molecular weight of the silicone-containing component. Useful silicone-containing components preferably include polymerizable functional groups such as acrylate, methacrylate, acrylamide, methacrylamide, vinyl, N-vinyl lactam, N-vinylamide, and styryl functional groups.

The functionalized media insert 150 can be partially or entirely embedded in the hydrogel portion 155; or in some embodiments the functionalized media insert 150 can be placed onto the hydrogel portion. In some embodiments, the media insert 150 can be used to encapsulate and act as a substrate for electronic elements and, in some embodiments, energization elements. In some embodiments, the electronic elements, including for example the active agent release system 105, can preferably be located outside of the optical zone 120, such that the device does not interfere with a user's sight. The active agent delivery system 105 may be powered through an external means, energy harvesters, and/or energization elements contained in the ophthalmic device 150. For example, in some embodiments the power may be received using an antenna (not shown) receiving RF signals that is in communication with the active agent release system 105.

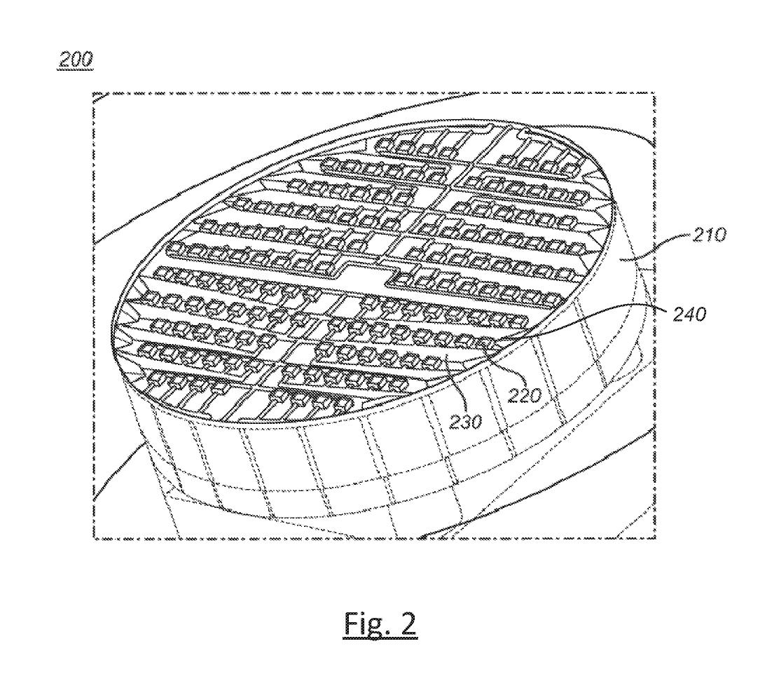

Referring now to FIG. 2, a close up representation of a surface of semiconductor device 210 with the containment array 200 of containment cells 220 forming part of the active agent release system 105 is depicted. The semiconductor device 210, e.g., silicon piece, can include circuitry for the control of the containment array 200 and to ensure that each containment cell can be engaged by an activation element 240 to cause the dispensing of an active agent. Each containment cell can be a reservoir-shaped region of the silicon, and may be filled with the active agent, e.g., one or more of a lubricant, a saline, a solvent, a pharmaceutical, and a nutraceutical, during assembly. Interconnect metallurgy may be used to define a matrix of regions overlying at least of portion of a surface of each of the containment cells. The interconnect metallurgy can be located on the same side of the silicon as the circuits. Containment cell 220 can include a metal cap bonded in a manner such that it is under stress and contains the active agent. The metal cap can include one or more biocompatible metals including, for example, gold, titanium, nickel, stainless steel, cobalt-chromuim, and nitinol. Other biocompatible non-permeable metals including binary metals may be used. According to some aspects of the disclosure, through the bonding of the metal cap to the silicon, by means of how it is assembled or the binary shape material, the metal cap can remain under stress while it is bonded. The assembly and bonding of the metal cap to the silicon piece may include, for example, braiding, welding, gluing, and the like.

The activation element 240 can include interconnects 230 positioned to be configured in such a manner that current flow may be directed to a portion or across the metal cap under stress on demand. This current flow and the stress which the metal cap is under can cause the metal cap to fold, thereby exposing the active agent to the surrounding environment. The folding can allow innocuous delivery of the active agent since, unlike some other systems, the metal does not have to melt or evaporate to expose the underlying contents of the containment cell. In some embodiments, the cap is manufactured so that the metal cap folds towards the inside of the containment cell. This can further prevent the metal cap from interfering with the surrounding cells and may assist ensuring that the active agent is dispensed accordingly. In other embodiments, the metal cap may be small enough that the folding does not produce an adverse effect to the surrounding cells and the direction of the folding does not affect the surrounding cells.

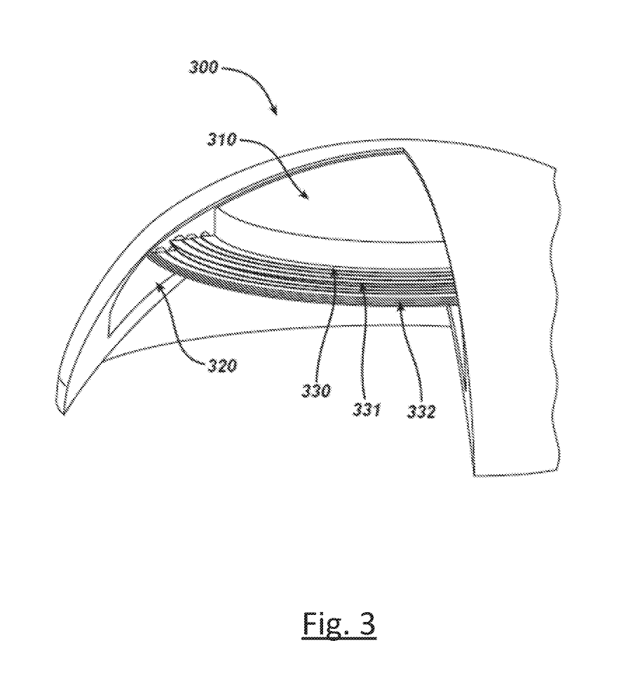

Referring now to FIG. 3, a diagrammatic representation of another exemplary energized ophthalmic device including both optics and the active agent release system is depicted. In particular, a three dimensional cross section representation of an exemplary ophthalmic lens 300 including a functionalized layer media insert 320 configured to include the active agent release system on one or more of its layers 330, 331, 332, is illustrated. In some embodiments, the media insert 320 surrounds the entire periphery of the optical zone 310 of the ophthalmic lens 300. Media insert 320 may be in the form of a full annular ring, a partial annular ring, or other shapes that still may reside inside or on the hydrogel portion of the ophthalmic lens 300 and be within the size and geometry constraints presented by the ophthalmic environment of the user.

Layers 330, 331, and 332 illustrate three of the numerous layers that may be found in an exemplary media insert 320 including a stack of functional layers. In some embodiments, for example, a single layer may include one or more of: active and passive components and portions with structural, electrical or physical properties conducive to a particular purpose, including the communication system functions described herein. Furthermore, in some embodiments, a layer 330 may include an energy source, such as, one or more of: a battery, a capacitor, and a receiver within the layer 330. Layer 331 then, in a non-limiting exemplary sense, may include microcircuitry in a layer that detects actuation signals for the ophthalmic lens 300. In some embodiments, a power regulation layer 332, may be included that is capable of receiving power from external sources, charges the battery layer 330 and controls the use of battery power from layer 330 when the ophthalmic lens 300 is not in a charging environment. The power regulation may also control signals to an exemplary active lens, demonstrated as item 310 in the center annular cutout of the media insert 320.

An energized lens with an embedded media insert 320 may include an energy source, such as an electrochemical cell or battery as the storage means for the energy and in some embodiments, encapsulation, and isolation of the materials including the energy source from an environment into which an ophthalmic device is placed. In some embodiments, a media insert 320 can also include a pattern of circuitry, components, and energy sources. Various embodiments may include the media insert 320 locating the pattern of circuitry, components and energy sources around a periphery of an optic zone through which a wearer of an ophthalmic lens would see, while other embodiments may include a pattern of circuitry, components, and energy sources which can be small enough to not adversely affect the sight of the ophthalmic lens wearer and therefore the media insert 320 may locate them within, or exterior to, an optical zone.

Reference has been made to electronic circuits making up part of the componentry of ophthalmic devices incorporating the active agent release system. In some embodiments according to some aspects of the invention, a single and/or multiple discrete electronic devices may be included as discrete chips, for example, inside, on, or positioned near the media insert. In other embodiments, the energized electronic elements can be included in the media insert in the form of stacked integrated components.

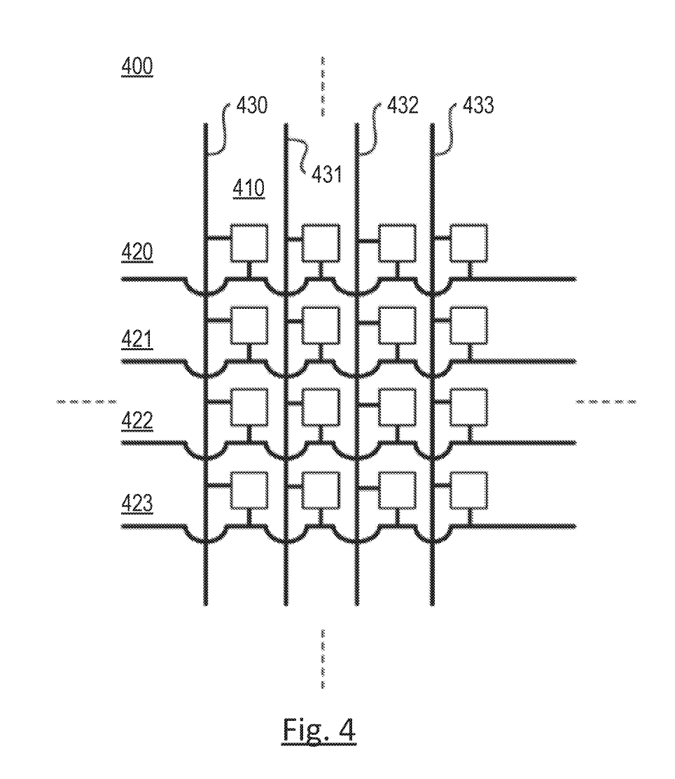

Referring to FIG. 4, item 400, depicts an exemplary routing of metal lines to allow for the connection of individual metal caps on top of the containment array. The individual metal caps are shown as the array of squares, one example of which is item 410. Although depicted as squares in FIG. 4, other shapes are contemplated. Depending on the actual size of the entire array there may be numerous additional cells that are not depicted in this figure. Also shown in the figure are a combination of four horizontal lines (420, 421, 422 and 423), which for illustration purposes and in a similar fashion to routing for memory cells may be classified as “word lines.” There are also 4 vertical lines (430, 431, 432 and 433) depicted as a subset of the “bit lines” in the array. By arranging the cells into a configuration where bit lines and word lines are capable of addressing all the containment cells, an efficient scheme may be realized. For example, if it were desirable to release the medicament located under cell 410, then current may be allowed to flow through item 430, then through the metal cap 410, and then out 420. As described in other parts of the disclosure, this controlled delivery can provide for the release of one or more type of various active agents when they are most needed.

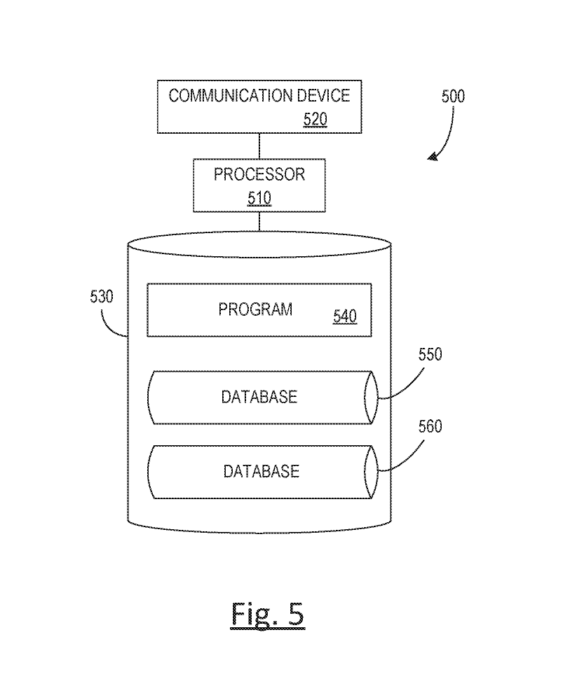

Referring now to FIG. 5, a schematic diagram of an exemplary micro-processor that may be used to implement some aspects of the present invention is illustrated. The micro-processor which can be referred to as the controller 500 can include one or more processor(s) 510, which may include one or more processor components coupled to a communication device 520. In some embodiments, a controller 500 can be used to transmit energy to the energy source placed in the ophthalmic lens and for the dispensing of the one or more active agents.

In some embodiments, the processor(s) 510 can be coupled to a communication device 520 configured to communicate energy via a communication channel. The communication device may be used to electronically communicate with components within the media insert, for example. The communication device 520 may also be used to communicate, for example, with one or more controller apparatus or programming/interface device components.

The processor 510 is also in communication with a storage device 530. The storage device 530 may include any appropriate information storage device, including combinations of magnetic storage devices, optical storage devices, and/or semiconductor memory devices such as Random Access Memory (RAM) devices and Read Only Memory (ROM) devices.

The storage device 530 can store a program 540 for controlling the processor 510. The processor 510 performs instructions of a software program 540. For example, the processor 510 may receive information descriptive of a sensed ophthalmic condition, component placement, a timer, and the like. The storage device 530 can also store ophthalmic related data in one or more databases 550 and 560. The database may include, for example, predetermined surrounding environment condition thresholds, sensed data, and specific control sequences for controlling components, e.g., controlling energy between components. The database may also include parameters and controlling algorithms for the control of the release system that may reside in the ophthalmic device as well as data and/or measured feedback that can result from their action. In some embodiments, that data may be ultimately communicated to/from an external reception device.

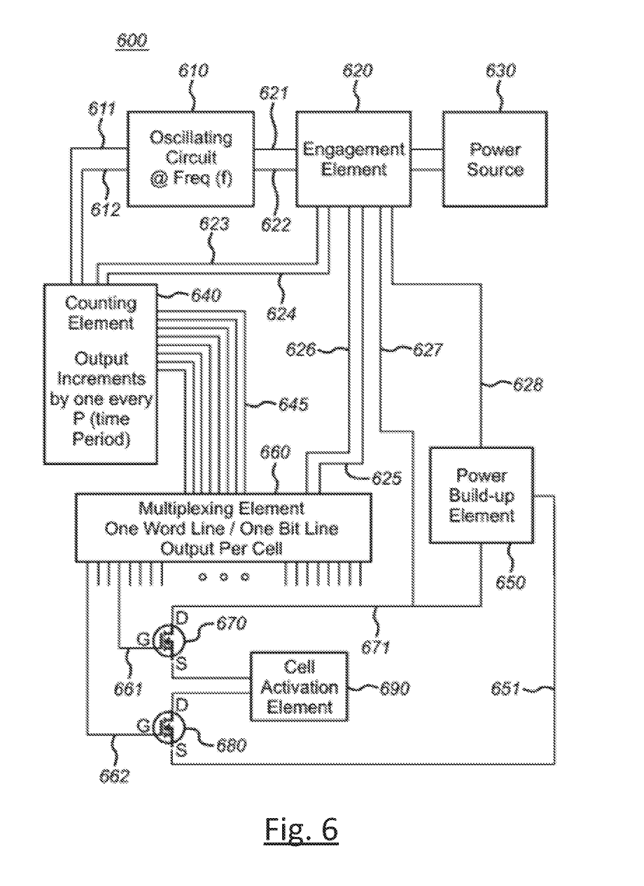

Referring now to FIG. 6, an exemplary design 600 for interconnections to individual active agent containment cells is depicted, including timing and control circuits that can be used to activate a particular containment cell. In some embodiments, the circuit can include a power source 630. This power source may be an alkaline battery or an energy receptor (e.g., an antenna). The power may be routed from the power source to the engagement element 620. This element may be set to an “on” state when the ophthalmic device is placed into the eye environment. When it is set to an on state, then the power source may be routed through engagement element 620 and out to other circuit elements. Items 621 and 622 may be the routing to an oscillating circuit element 610. Items 623 and 624 may be the routing to a counting element 640. Items 625 and 626 may be the routing to a multiplexing element 660. And, items 627 and 628 may be the routing to a power build-up element 650.

Once the power is engaged in the energized ophthalmic device, the oscillating circuit may begin its oscillation at a particular frequency. The output of element 610 may be passed to the counting element 640 via items 611 and 612. The counting element 640 may have a duty cycle that counts for a certain number of cycles on the input line 612. In an exemplary sense, the combination of the frequency of oscillation and the count required before the output of the counting element increments by one may correspond to a specified time period (e.g., 2 hours). Therefore, in this example, every two hours the output of counting element 640 will be increased by one count. This count may be encoded into an eight bit number which is passed from the counting element 640 to the multiplexing element 660 through the data bus 645.

The multiplexing element 660 may receive the eight bit number and decode this number into a unique combination of a first word line 661 and a first bit line 662. When a particular word line is activated (e.g., line 661), it may turn on a power transistor 670 to current flow. The bit line 662 may turn on a power transistor 680. As was shown in FIG. 5, a combination of bit line and word line may address a unique array element in the containment array 400. When the power transistors are engaged, power may be routed from a power build up element 650 through line 651, then through cell activation element 690, and out of line 671. When the current runs through the cell activation element, or the cell activation element is otherwise engaged, the metal cap may fold out of the way, thereby exposing the active agent contained in the respective containment cell to the surrounding environment.

There may be numerous variations that are possible with this type of circuit. For example, it may be possible to use the charge up time of item 650 in concert with a resistive element to determine the timing from one cell exposure to another replacing the need for an oscillating circuit. Other variations that may be possible include, for example, that the multiplexing element addresses a unique output line for every containment cell. In addition, the circuit may activate a single cell at a particular time period. It may be apparent to one skilled in the art that various diversity may derive from electronically controlled delivery; including in a non-limiting sense delivering discrete doses of active agent from containment cells at different programmed rates, and programming multiple containment cells to deliver doses at a particular time period.

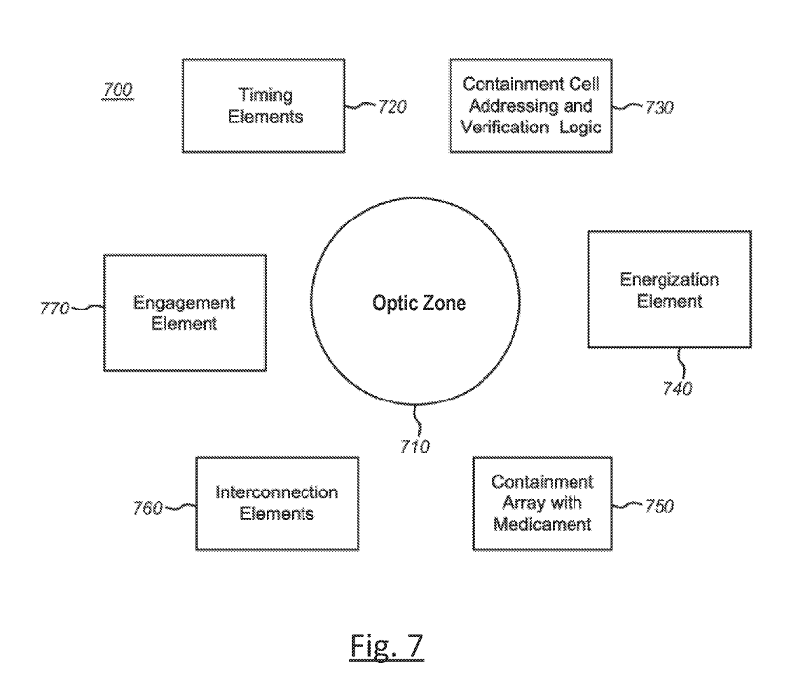

Referring now to FIG. 7, a block diagram showing components of an exemplary ophthalmic device with an energized containment array is depicted. In particular, and as mentioned in the previous paragraphs, the formed energized ophthalmic device may contain all of the elements shown at 700 as items optic zone 710, timing elements 720, containment cell addressing and verification logic 730, energization element 740, containment array with medicament 750, interconnection elements 760, and engagement or activation element 770. It may be instructive to consider how these elements may function in practice.

An ophthalmic device may be placed on the anterior surface of the eye. In the process of placing the ophthalmic device in the eye the engagement element 770 may be set to an “on” state. This can allow for power to be sent from an energization element 740, to all the other elements. The timing elements 720 (e.g., oscillator and counting elements), may begin to start counting. After a preprogrammed time has elapsed, e.g., two hours, the counting element may index a position. The multiplexer 730 may then configure a single word line and a single bit line to conduct current. This combination will define an array element within the containment array 750 and the current flow may cause the metal cap to fold, thereby uncovering the active agent of this first containment cell. In some embodiments, opening of the containment cell may allow for tear fluid to enter the cell and dissolve a dissolvable active agent away. Accordingly, the active agent may be quickly released into the eye environment in a well regulated manner. A second counter may also be used, for example, to disengage the multiplexer after a certain count has been reached, so that the battery element is not discharged should a failure cause a constant current draw.

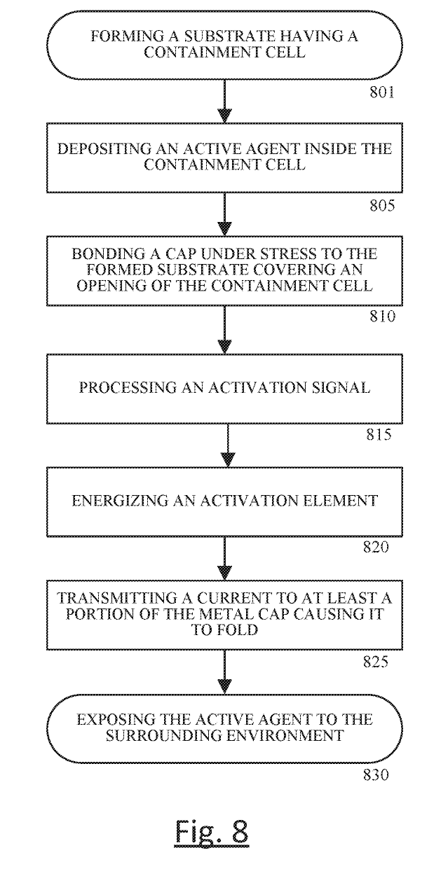

Referring now to FIG. 8, a flow chart with exemplary method steps that can be used to carry out some aspects of the present invention is depicted. Beginning at step 801, a substrate having one or more containment cells can be formed. As previously described, the substrate can include a silicon wafer with a series of reservoir-shaped containment cells formed therein. Each containment cell may be assembled, for example, with an activation element in communication with an energy source and one or more sensor(s). At step 805, an active agent can be deposited into each of the containment cells. The active agent is preferably in the form of a concentrated solution that can be diluted by a solution including, for example, tear film. The concentration of the solution can be selected to achieve a desired dosing level. After depositing the active agent into a containment cell, at step 810 a cap can be bonded under stress onto a containment cell surrounding surface, such that the containment cell can be sealed. In some embodiments, the opening sealed by bonded metal cap under stress may be the same opening used to deposit the active agent during assembly.

At step 815, an activation signal can be processed by a micro-processor in communication with an activation element. The activation signal may be received from one or more sensor(s), an oscillating element, an internal or external input from a user, a device in wireless communication, and the like. For example, a user may input a command for the activation signal to be processed using a device in wireless communication, through an antenna, with the micro-processor of the ophthalmic device. In some embodiments the collection of data may occur in the microprocessor of the ophthalmic device, using one or more sensors, and transmitted to a device in wireless communication for external data analysis. The device may then process the data received, and sometimes additional data from one or more other external sources and/or user inputs, to determine and send an activation signal when the dispensing of the active agent is needed. As previously mentioned, the device can include one or more of: a smart phone, a tablet, a personal computer, a remote transmitter, and a medical drug delivery device, and the like. Transmission of information between the device and the micro-processor of the ophthalmic device can occur wirelessly, for example, via any low power RF frequency.

At step 820, energization of the activation element can occur. Upon energization, at step 825, a current of a pre-determined range can be delivered to a portion of the metal cap bonded under stress, causing it to fold. Accordingly at step 830, the active agent is then exposed to the surrounding environment as previously described. The range of the current can vary, as it will be apparent to one skilled in the art from the present disclosure, depending on the thickness of the metal cap, the type of metal, the method of assembly, and/or the size of the metal cap.

Many features and advantages of the invention are apparent from the detailed specification, and thus, it is intended by the appended claims to cover all such features and advantages of the invention which fall within the true spirit and scope of the invention. Further, because numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A method of dispensing an active agent comprising:
forming a substrate having one or more containment cells;
depositing one or more active agents into at least one of the one or more containment cells;
forming a hermetic seal over an opening of at least one of the one or more containment cells by bonding a biocompatible metal cap under stress to a surface of the substrate; and
providing an activation element configured to conduct an electrical current from an energy source to at least a portion of the biocompatible metal cap causing the biocompatible metal cap to fold and thereby expose the active agent to a surrounding environment.

2. The method of claim 1, additionally comprising:
providing a micro-processor in connection with an antenna and the energy source; and
receiving, using the antenna, a wireless signal from a wireless device used to generate an activation signal for the energizing of the activation element.

3. The method of claim 1, wherein the active agent can include one or more of: a lubricant, a saline, a solvent, a vitamin, and a medicament.

4. The method of claim 1 additionally comprising:
encapsulating at least part of the substrate in a hydrogel.

5. The method of claim 1 additionally comprising:
encapsulating the energy source in a media insert configured to be positioned in an ophthalmic device and supporting the substrate and the activation element.

6. The method of claim 1, wherein the energy source is an energy receptor antenna in electrical communication with said activation element.

7. The method of claim 1, additionally comprising:
providing a micro-processor in connection with one or more sensors and the energy source; and
generating an activation signal for energizing the activation element based upon one or more of said sensors detecting a pre-defined parameter.

8. A method of manufacturing an ophthalmic device with an active agent release system comprising:
forming a substrate having one or more containment cells;
depositing one or more active agents into at least one of the one or more containment cells;
forming a hermetic seal over an opening of at least one of the one or more containment cells by bonding a biocompatible metal cap under stress to a surface of the substrate; and
providing an activation element configured to conduct an electrical current from an energy source to at least a portion of the biocompatible metal cap causing the biocompatible metal cap to fold and thereby expose the active agent to a surrounding environment.

9. The method of claim 8, additionally comprising:
providing a micro-processor in connection with an antenna and the energy source.

10. The method of claim 8, wherein the active agent can include one or more of: a lubricant, a saline, a solvent, a vitamin, and a medicament.

11. The method of claim 8, additionally comprising:
encapsulating at least part of the substrate in a hydrogel.

12. The method of claim 8, additionally comprising:
encapsulating the energy source in a media insert configured to be positioned in an ophthalmic device and supporting the substrate and the activation element.

13. The method of claim 1, wherein the energy source is an energy receptor antenna in electrical communication with said activation element.

14. The method of claim 8, additionally comprising:
providing a micro-processor in connection with one or more sensors and the energy source.
